(19) United States
(12) Patent Application Publication
(10) Pub. No.: US 2003/0078653 A1
(43) Pub. Date: Apr. 24, 2003

(54) Tissue engineered mitral valve chordae and methods of making and using same

(76) Inventors: Ivan Vesely, Cleveland Heights, OH (US); Kathryn Jane Grande-Allen, Euclid, OH (US)

Correspondence Address: BENESCH, FRIEDLANDER, COPLAN & ARONOFF LLP, ATTN: IP DEPARTMENT DOCKET CLERK, 2300 BP TOWER, 200 PUBLIC SQUARE, CLEVELAND, OH, 44114, US

(21) Appl. No.: 10/174,120

(22) Filed: Jun. 17, 2002

Related U.S. Application Data
(60) Provisional application No. 60/298,547, filed on Jun. 15, 2001.

(51) Int. Cl. A61F 2/24; A61F 2/02

(52) U.S. Cl. 623/2.16; 623/23.72; 623/918

(57) ABSTRACT

A tissue equivalent and method of making and using same is provided herein. The tissue equivalent disclosed herein is particularly useful in the repair or replacement of mitral valve chordae, and is prepared by combining collagen with living tissue cells to form a collagen gel and controlling shrinkage of the collagen gel to cause collagen fibrils in the collagen gel to align along a single axis in an unbranched configuration or multiple paths in a branched configuration.

### CROSS-REFERENCE TO RELATED APPLICATIONS

This application claims the benefit of U.S. Provisional Application No. 60/298,547 filed on Jun. 15, 2001.

### BACKGROUND AND SUMMARY OF THE INVENTION

Mitral valve repair is a common surgical technique used to treat mitral valve dysfunction and regurgitation. Valves with torn marginal chordae are typically repaired by excising the prolapsing valve segment. Mitral valve repair is a very difficult surgical procedure that is currently performed only at select sites by trained and experienced surgeons. Even fewer surgeons attempt complete chordal replacement. In cases where sections with damaged chordae cannot be excised, artificial chordae have been used. Expanded polytetrafluoroethylene (ePTFE) is the most commonly used material for fabrication of artificial chordae for mitral valve repair. The disadvantages of ePTFE are that: (i) it is not available in branching configurations, (ii) it has mechanical properties unlike natural mitral valve chordae, (iii) its mechanical properties change with time, and (iv) it can cause a foreign body reaction and local inflammation.

Other materials used to fabricate artificial tissue such as chordae are silk and nylon. However, like ePTFE, these synthetic materials can cause a foreign body reaction and local inflammation. Moreover, these materials do not have the mechanical properties needed to properly extend and cushion the impact of mitral valve closure. Tissue-engineering technologies offer the promise of creating biological materials with the appropriate physical, mechanical and biological properties.

Since load-bearing connective tissues are composed primarily of Type I collagen, tissue equivalents fabricated from collagen are a logical choice. Collagen is a natural cell substrate and provides biological responses similar to those of natural chordae. Tissue-engineering principles can be applied to fabricate mitral valve chordae in vitro using directed collagen gel shrinkage. Collagenous tissues or tissue equivalents with a desired microstructure can be generated. If collagen gel is mechanically constrained and shrinkage is prevented in a particular direction, the collagen fibrils in the gel align in the direction of constraint. This allows for the fabrication of highly aligned, compacted collagenous tissue equivalents. This principle has been used to fabricate materials for blood vessels, tendon and even heart valves.

What is needed is a tissue engineered mitral valve chordae that may be branched or unbranched and a method of making and using the same. Current technology only provides for one-dimensional tissue engineered constructs (i.e., single cord like structures). However, the present invention provides for a two-dimensional tissue engineered construct (i.e., branched mitral valve chordae) and a three-dimensional tissue engineered construct for mitral valve chordae or other applications within the body.

### SUMMARY OF THE INVENTION

The present invention provides a novel tissue equivalent particularly useful in the repair and replacement of mitral valve chordae. More particularly, the present invention relates to a tissue equivalent made from fibrillar collagen and living tissue cells wherein the collagen fibrils are compacted and generally aligned along a single axis in the case of an unbranched mitral valve chordae or aligned along multiple paths in the case of a branched mitral valve chordae. Preferably, the living tissue cells are balanced and fortified with nutrient medium. More preferably, trace elements, such as Mg²⁺, Zn²⁺, Fe²⁺and Mn²⁺; amino acids, vitamins and growth factors are added to fortify the living tissue cells. The tissue equivalents of the present invention can be branched or unbranched allowing for flexibility in the manner in which they are used. The tissue equivalent provided herein is also useful for repairing and replacing blood vessels, tendon and heart valves.

In one embodiment, a connective tissue equivalent is comprised of a body comprising collagen fibrils wherein the body has a proximal portion and a distal portion. The body terminates into at least two ends at the distal portion and the body terminates into at least one end at the proximal end wherein the body and ends have collagen fibrils oriented along an axis of alignment. The body may further comprises tissue cells embedded within the collagen fibrils. The distance along the body between one of the at least two ends at the distal portion and the end at the proximal end defines a first path such that a portion of the collagen fibrils are generally oriented along the direction of the first path. The distance along the body between the other of the at least two ends at the distal portion and the end at the proximal end defines a second path such that a portion of the collagen fibrils are generally oriented along the direction of the second path. The at least two ends at the distal portion are suitable for attachment to a tissue body such as a leaflet suturing strip. The end at the proximal portion is suitable for attachment to a tissue body such as a papillary muscle pad.

In another embodiment, a tissue equivalent comprising a construct comprised of collagen fibrils and living tissue cells embedded within the collagen fibrils, the construct including a body that extends into a first arm defining a first path and a second arm defining a second path. A portion of the collagen fibrils may be generally oriented in a direction substantially parallel to the direction of the first path at any given location along the first path, while a portion of the collagen fibrils may be generally oriented in a direction substantially parallel to the direction of the second path at any given location along the second path. The construct may include a proximal portion and a distal portion wherein the first arm terminates into a first end at the distal portion and the second arm terminates into a second end at the distal portion and the body terminates into a third end at the proximal end. The first and second ends at the distal portion may be suitable for attachment to a tissue body such as a leaflet suturing strip. The third end at the proximal portion may be suitable for attachment to a tissue body such as a papillary muscle pad. The tissue equivalent may further comprise a third arm that extends from the body thereby defining a third path.

In another embodiment, a connective tissue equivalent comprises a body having a plurality of arms extending from the body and the body and plurality of arms comprised of collagen fibrils having living cells embedded therein. The connective tissue equivalent may further comprise a tissue equivalent body in communication with the plurality of arms.

In another embodiment, a tissue engineered mitral valve chordae is comprised of a construct formed of collagen fibrils wherein the construct includes a body having a plurality of arms extending therefrom and the construct having mechanical integrity substantially similar to natural chordae. The mechanical integrity may be selected from the group consisting of extensibility, stiffness, strength, flexibility, pliability, and combinations thereof.

The present invention also provides a method for fabrication of collagenous tissue equivalents with compacted collagen fibrils generally aligned along along a single axis in the case of an unbranched mitral valve chordae or aligned along multiple paths in the case of a branched mitral valve chordae. More particularly, the present invention relates to a method of producing a collagenous tissue equivalent by combining diluted collagen and living tissue cells to form a collagen/cell suspension. The collagen/cell suspension is then placed in a mold under conditions allowing formation of a collagen gel with living tissue cells dispersed therein. The mold is adapted to mechanically constrain the collagen gel and inhibit shrinkage in a particular direction. The collagen gel is then maintained under conditions that allow contraction of the collagen gel thereby forming a tissue equivalent. Preferably the living tissue cells are fortified with nutrient medium prior to combining the cells with the collagen. Trace elements such as Mg²⁺, Zn²⁺, Fe²⁺and Mn²⁺; amino acids such as Met, Cys and α-KG; vitamins such as C and B complex; and growth factors may be added to the cells prior to combining the cells with collagen. By varying the manner in which the collagen gel is constrained as it contracts, one can vary the orientation of the collagen fibrils and the amount of extracellular matrix produced by the entrapped tissue cells. Further, the mechanical strength and biological properties of the tissue equivalent can be controlled by varying cell seeding density, initial collagen concentration, cell passage, serum concentration and culture time. By varying these parameters, tissue equivalents having properties similar to native tissue can be fabricated.

The present invention also provides a mold for the fabrication of collagenous tissue equivalents with compacted collagen fibrils. The mold of the present invention is constructed with means for attaching a collagen gel to the inner walls of the mold to cause the collagen fibrils to align in a direction transverse to the direction of attachment upon contraction. Preferably, the mold of the present invention is rectangular in shape for unbranched chordae or “Y” shaped for branched chordae. The mold can be constructed to allow for contraction along more than one axis or path.

### BRIEF DESCRIPTION OF THE DRAWINGS

FIG. 1 illustrates a mold for fabricating a tissue engineered construct according to the present invention;

FIG. 2 illustrates a rectangular shaped well 215 for fabricating an unbranched mitral valve chordae according to the present invention;

FIG. 3 illustrates a “Y” shaped well 315 for fabricating a branched mitral valve chordae according to the present invention;

FIG. 4A illustrates a “Y” shaped tissue engineered construct that is two-dimensional;

FIG. 4B illustrates a “Y” shaped tissue engineered construct that is three-dimensional because one arm of the “Y” shaped construct extends into the Z-dimension;

FIG. 5A illustrates an isolated branched chordae formed fabricated according to the present invention.

FIG. 5B illustrates examples of the manner in which tissue equivalents according to the present invention can be used in the repair and reconstruction of mitral valve chordae;

FIG. 6 illustrates the evolution of the collagen gel into the tissue equivalent according in the present invention over a period of time from 2 hours to 50 days; and

FIG. 7 illustrates a “Y” shaped or branched tissue engineered construct according to the present invention.

### DETAILED DESCRIPTION OF THE INVENTION

A tissue equivalent is defined herein as a material or construct which is formed in vitro with living cells and proteinaceous fibers and has mechanical and physiological properties similar to in vivo oriented tissue. Preferably, the tissue equivalent is oriented to thereby increase mechanical strength along the axis of alignment. These tissue equivalents may be used in the fabrication of a large number of tissue engineered material including, but not limited to, mitral valve chordae, suturing materials, blood vessels, tendon, connective tissue, and heart valves.

An initial step in the formation of a tissue equivalent comprises forming a collagen gel having connective tissue cells dispersed therein. The collagen useful in forming the gel can be extracted from various collagen-containing animal tissue. Examples of possible collagen-containing tissue are tendon, skin, cornea, bone, cartilage, invertebral disc, cardiovascular system, basement membrane and placenta. According to the present invention, any collagen may be used including, but not limited to, type I, II, or III collagen. Conditions whereby collagen can be extracted from are: 1) low ionic strength and neutral buffer; 2) weak acid solution; and 3) partial pepsin digestion followed by extraction in acid solution. For example, the collagen can be derived by acid extraction followed by salt precipitation of rat tail collagen from acid solution.

The connective tissue cells useful to contract the collagen fibrils in the formation of an a tissue equivalent can be obtained from various mammalian sources (e.g., bovine, porcine, human, canine, and rat). Examples of possible connective tissue cells are fibroblasts, smooth muscle cells, striated muscle cells and cardiac muscle cells. The connective tissue cells used in the method of the present invention were rat aortic smooth muscle cells and bovine chordae fibroblasts, but other types of connective tissue cells may be employed.

The isolated collagen and connective tissue cells can be cultured in a medium which provides nutrients to support cell growth, for example, Dulbecco's Modified Eagle Medium (DMEM). Additional components can be added to the medium to enhance collagen and cell growth and viability, for example fructose (in the absense of glucose), ascorbic acid, TGF-β (a growth factor), and gentamicin (an antibiotic). Other nutrients may include trace elements (e.g., Mg²⁺, Zn²⁺, Fe²⁺ and Mn²⁺); amino acids (e.g., Met, Cys and α-KG); and vitamins such as C and B complex. Growth factors may also be present in the tissue equivalents disclosed herein.

In the formation of a collagen gel, the mixture of collagen and connective tissue cells in a media as described above, is placed in a biocompatible container in which cells can be cultured, such as a petri dish. The dish can be coated with a water repellant to retard cell adhesion, such as organosilane.

At a slightly elevated temperature, the mixture of collagen and connective tissue cells will gel, corresponding to the precipitation of collagen molecules into fibrils. For example, with the collagen and fibroblasts used herein, warming of the mixture to about 37° C. is sufficient to induce collagen precipitation. The gels are then maintained under standard cell culture conditions well established in the art, suitable for contraction of the gel by the connective tissue cells. Over time, the cells consolidate and organize the collagen fibrils producing macroscopic contraction of the gel. The tissue equivalent is formed as the embedded connective tissue cells contract the gel by attaching to and pulling together collagen fibers to form a collagen structure or construct. These collagen structures can then be crosslinked in preparation for use in humans, or, if manufactured from autologous collagen and autologous cells, implanted as-is.

Structural members (also known as holders or anchors) which can be used to restrain the contraction process of the collagen fibrils by the connective tissue cells can be of various shapes, diameter and height and can be easily accommodated within the dimensions of the culture dish. The structural members can be spaced a predetermined distance apart to provide an axis along which the cells can align the collagen fibrils. For example, the structural members can be cylindrical posts (vertical), cylindrical rods (horizontal), spherical objects such as pellets, and rectangular bars. Further, the structural members can be formed of metal such as stainless steel or a biocompatible material, such as polyethylene or hydroxyapatite. Preferably, the structural members are porous where the pore size of the structural members can be about a few hundred microns to allow for cell attachment and growth within and around the member. If the structural member is made of a metal, it is preferred that the metal is wrapped or covered with a porous material to permit cell attachment. Preferably, the porous material is glass fiber. The pattern of cell alignment is consistent with the belief that cells are able to orient by matrix rigidity. As cells exert traction on the collagen matrix, the matrix becomes consolidated in the unconstrained axes. However, along the axis between the two rigid structural members, the cells align the matrix which stiffens, and provides cells an orientation cue. In the periphery of the oriented tissue-equivalent, cell alignment is not observed due to relatively unrestrained matrix compaction in all dimensions. In the center of the tissue equivalent, where matrix compaction is rigidly constrained along one axis, uniform cell alignment is observed. These results suggest that to obtain a tissue equivalent that is uniformly oriented, the initial diameter of the reconstituted collagen gel should be small relative to the distance between the two structural members.

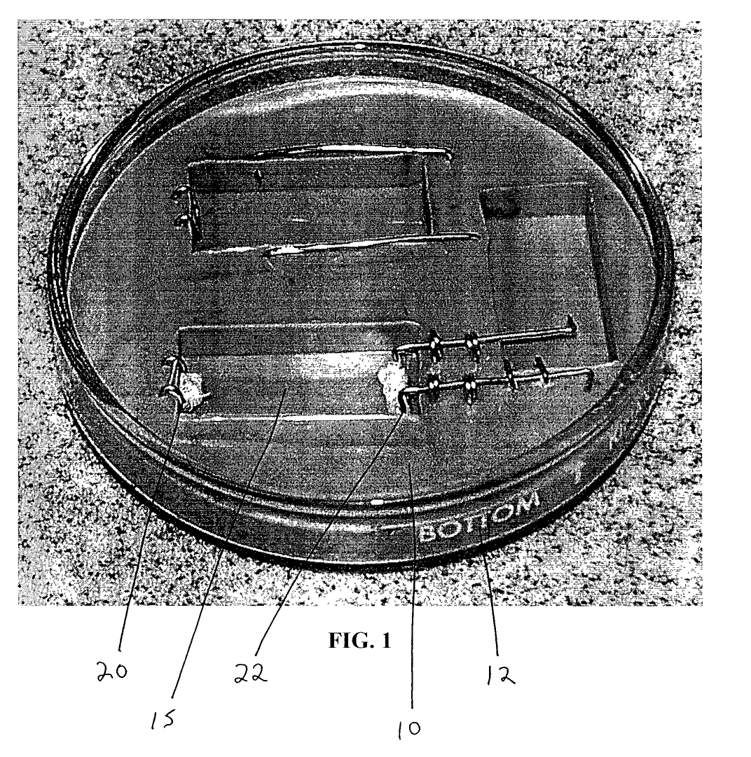

The apparatus used to fabricate the tissue equivalent according to the present invention comprises a rubber silicone mold for receiving the gel. Preferably, the rubber silicone mold 10 may be fitted into a 100 mm diameter petri dish 12 as shown in FIG. 1. The mold includes a recessed well 15 that may be cut-out in any size or shape depending on the desired tissue equivalent application. The well 15 includes at least two structural members 20, 22 for restraining the gel as described above.

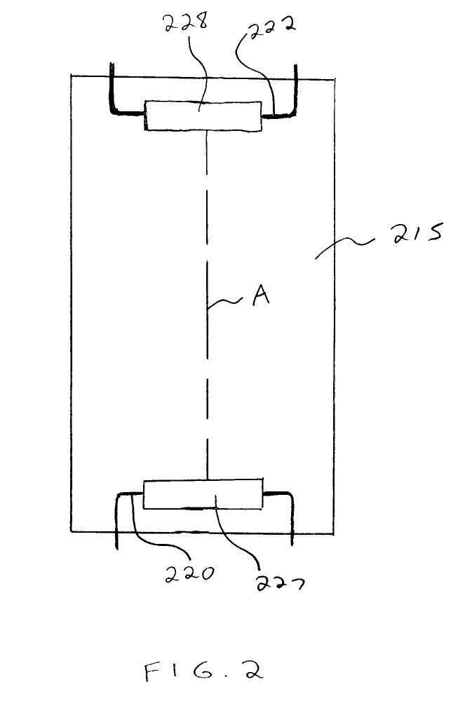

To fabricate an unbranched mitral valve chordae, the mold preferably includes a rectangular shaped well 215 as shown in FIG. 2. The well includes structural members 220, 222 positioned at each end of the rectangular shaped well a fixed distance apart. The structural members 220, 222 may be positioned at each end of the well by any means known in the art. Preferably, the structural members 220, 222 are cylindrical rods that are horizontal and wrapped with glass fiber 227, 228. An axis (A′) is defined as the line joining the two rods (i.e., 220 and 222) along which the cells can align.

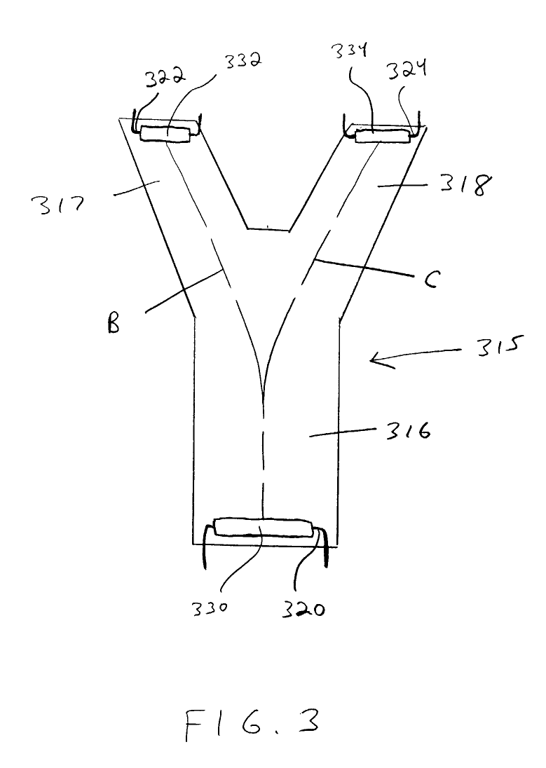

To fabricate a branched (i.e., two branch) mitral valve chordae, the mold preferably includes a “Y” shaped well 315 as shown in FIG. 3. The “Y” shaped well 315 has a body 316 that extends into two arms 317, 318. The well includes structural members 320, 322, 324 positioned at each end of the “Y” shaped well at fixed distances apart. The structural members 320, 322, 324 may be positioned at each end of the well by any means known in the art. Preferably, the structural members 320, 322, 324 are cylindrical rods that are horizontal and wrapped with glass fiber 330, 332, 334. Two paths may be defined with respect to the “Y” shaped well 315 in the mold in which the cells can align. First, Path B′ may be defined as the line joining rods 320 and 322. Second, Path C′ may be defined as the line joining rods 320 and 324. One skilled in the art would also recognize that the branched mitral valve chordae may include three or more arms. In this case, the well would include a third arm extending from the body wherein the end of the third arm would include a fourth structural member for cell attachment. The key to a two-dimensional geometry is to ensure that tension is properly controlled during the shrinkage process so that the tissue engineered constructs do not tear away from the structural members during the early stages. As one skilled in the art would appreciate, different branching angles may be engineered with the use of appropriate molds. Also, in the case of the “Y” shaped well, it is possible to vary the widths of the structural members 320, 322, 324 to optimize the performance of the branched mitral valve chordae. The preferred ratio between the “parent” structural member 320 and the “daughter” structural members 322 and 324 is 2:1, but other ratios are possible depending on the application.

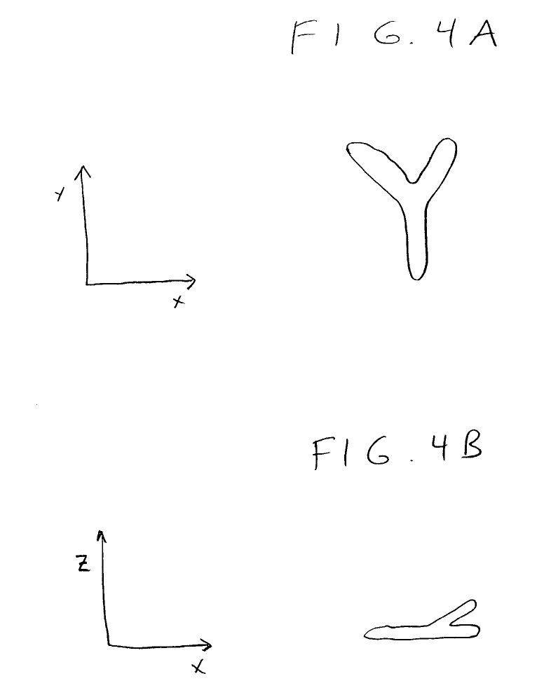

The present invention not only provides for a two-dimensional tissue equivalent (see FIG. 4A), but also provides for a three-dimensional tissue equivalent. To accomplish this, the structural members may be positioned in different planes in the Z-direction (i.e., raised or lowered with respect to each other) as shown in FIG. 4B. The contraction process will align or orient the connective tissue cells along the direction in which contraction is restrained and therefore would result in a three-dimensional tissue equivalent.

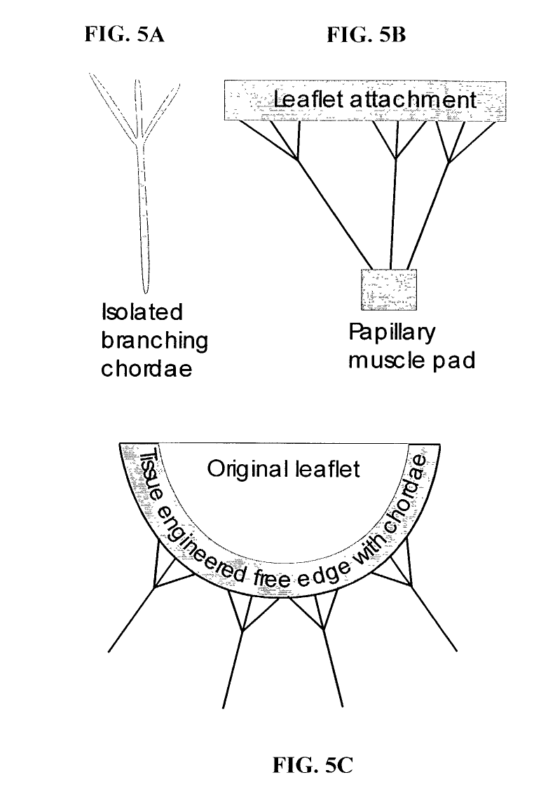

The tissue equivalents of the present invention can be used for a variety of surgical procedures involving repair or replacement of the mitral or tricuspid valve as shown in FIGS. 5A-5C. For example, these tissue equivalents can be used to replace individual mitral valve chordae. FIG. 5A depicts an isolated branched chordae formed from the tissue equivalent of the present invention. FIGS. 5B and 5C also illustrates examples of the manner in which such tissue equivalents can be used in the repair and reconstruction of mitral valve chordae as heretofore described. In this instance, the surgeon may use either a branched or unbranched chordae. The advantage of branched chordae is that the leaflet can be properly supported during mitral valve repair by multiple chordae (as illustrated in FIG. 5C), rather than at one or two points as is done conventionally.

The tissue equivalents of the present invention can also be used in valve repair to replace damaged chordae and to surgically reconstruct the leaflet free edge. With a branching network of chordae affixed to a leaflet suturing strip, the entire free edge of the leaflet can be surgically reconstructed. This gives the surgeon tremendous flexibility in repairing heavily diseased mitral valves that could not have been repaired previously, and would have needed to be completely replaced with a prosthesis. With a branching network of chordae affixed to a suturing strip, the entire free edge of the leaflet can be surgically reconstructed.

The tissue equivalents of the present invention can also be used to completely replace a valve with an artificial valve. Preservation of the valve chordae in valve replacement procedures is encouraged in order to preserve ventricular function. The tissue-engineered chordae and suturing-strip devices of the present invention can be used to augment the tethering of the ventricle in cases where the native chordae are insufficient. The suturing strips could be sewn to the valve annulus and the papillary muscles prior to implantation of the prosthetic valve. The present invention may also provide for a branched or unbranched chordae, (ii) complex, highly branched chordae attached to a leaflet suturing strip, and (iii) branched or unbranched chordae attached to a leaflet suturing strip at the upper border and a papillary muscle suturing strip at the lower border. These tissue equivalents are useful in the repair and replacement of mitral valve chordae.

Optimization of the cell-mediated, directed gel contraction process is an effective means of increasing the mechanical strength of the mitral valve chordae constructs made from the tissue equivalent of the present invention. Since proper orientation is likely responsible for the superior strength of our constructs, the configuration of the grips and mold are important parameters. Also, an appropriate, nutrition-balanced and fortified medium is effective in increasing collagen biosynthesis and improving the mechanical strength of the tissue equivalents of the present invention.

Leaflet and Papillary Muscle Pad

The present invention will be further understood by reference to the following non-limiting examples illustrating the preparation of the mitral valve chordae of the present invention. The present invention is not restricted to these examples.

### EXAMPLE 1

First, neonatal rat aortic smooth muscle cells (NRASMCs) were isolated by means known in the art. Then, segments of aorta were incubated with 2 ml of type II collagenase (2 mg/ml in DMEM/F12 (1:1) medium; Worthington Biomedical) for 10 minutes at 37° C. to remove the endothelium. The explants were then washed with PBS several times, minced into small pieces, transferred onto a sterile petri dish, and incubated in limited volumes of equal ratio of DMEM and F12, supplemented with 20% fetal bovine serum (Invitrogen, Carlsbad, Calif.), at 37° C. for one week to establish the primary culture. After reaching confluence, the cells were detached by trypsinization with 1 ml of 0.05% fresh trypsin containing 0.2% EDTA (Invitrogen, Carlsbad, Calif.), suspended in the above medium, and centrifuged at 1500 rpm. The obtained cell pellet was resuspended, counted, and seeded in 75 ml plates for passaging. Cells were stained with trypan blue and a hemocytometer was used to determine cell density and viability. Culture medium was changed twice a week. Prior to use, cells were detached from the culture dishes by trypsinization, counted, centrifuged, and added to the collagen suspension at a cell-seeding concentration of 1.0 million cells/ml.

Fetal bovine serum and Pen-Strep were thawed and added to the medium (5×DMEM/F12) to obtain a solution of 10% serum and 100-units/ml Pen-Strep. Sterile acid-soluble type I collagen (BD Biosciences, Rat tail; 3.94 mg/ml, 0.02 N acetic acid) was added to get an initial concentration of 2.0 mg/ml. The suspension was brought to physiological pH by the addition of 0.1 N NaOH and the cells were added. All mixing was done on ice.

This collagen-cell suspension was then pipetted into the rectangular shaped well 215 in the mold as described above and shown in FIG. 2 to fabricate an unbranched mitral valve chordae. The collagen-cell suspension in the well was incubated at 37° C. Within several minutes, the collagen gel formed and attached to the porous cylindrical rods at the ends of the wells. Within several hours, the collagen gel detached from the walls of the well and began to contract.

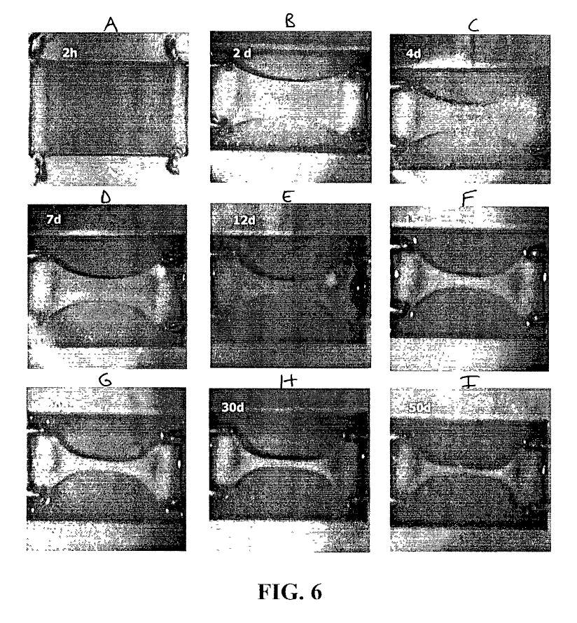

Contraction was rapid initially, eventually slowed down, but continued for up to 8 weeks. Culture medium was changed every 2 days. The rods restrained the contraction process of the gel by the cells to form a tissue equivalent between the rods having a “dumbbell shape”. The contraction process aligns or orients the connective tissue cells along the direction in which contraction is restrained which in this configuration is parallel to or along the axis joining the two rods (i.e., Axis A′). FIGS. 6A-6I depicts this contraction process after various time intervals. The original transparent gel became a dense, cylindrical construct as shown in FIG. 6I.

The final unbranched construct had the typical nonlinear stress/strain curve of tendinous materials, an extensibility of 10-15%, a stiffness of 13 MPa and failure strength of 1.9 MPa. Ultrastructural analyses have shown that the main reason for the good strength of the tissue equivalent constructs is the very high collagen fibril density. Because the constructs are relatively simple, one-dimensional collagen bundles, they compacted from two directions, producing an area shrinkage ratio greater than 99% (from an area of 324 mm² to less than 1 mm²). When fully compacted, the collagen fiber density visually approaches that of mitral valve chordae, with well-aligned collagen fibrils. Success has also been seen in inducing collagen fiber crimp in our constructs by controlling the tension applied to them during shrinkage, but clearly not to the same fidelity as occurs in chordae.

### EXAMPLE 2

The collagen-cell suspension was prepared in the identical manner as described above in Example 1. Once the collagen-cell suspension was prepared, it was pipetted into the “Y” shaped well as described above and shown in FIG. 3 to fabricate a branched (i.e., two branch) mitral valve chordae. The collagen-cell suspension in the well was incubated at 37° C. Within several minutes, the collagen gel formed and attached to the porous cylindrical rods at the ends of the wells. Within several hours, the collagen gel detached from the walls of the well and began to contract.

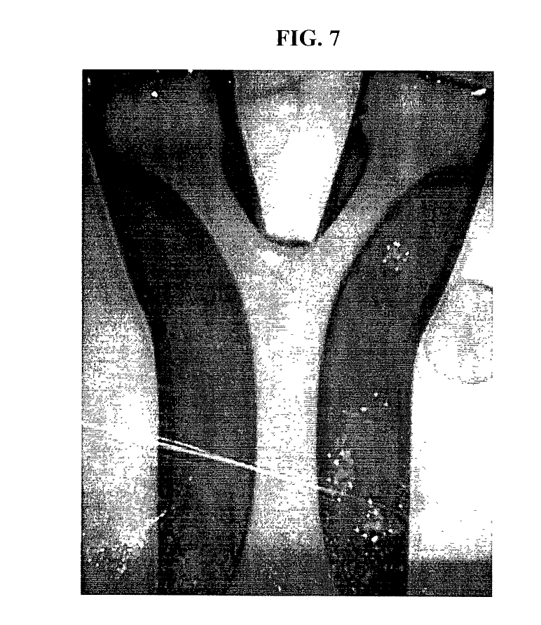

Contraction was rapid initially, eventually slowed down, but continued for up to 8 weeks. Culture medium was changed every 2 days. The rods in the well restrained the contraction process of the gel by the cells to form a tissue equivalent between the rods having a “Y” shape as shown in FIG. 7. The contraction process aligns or orients the connective tissue cells along the direction in which contraction is restrained. In this configuration, the connective tissue cells are aligned along a direction that is generally parallel to either Path B′ or Path C′.

### EXAMPLE 3

A series of experiments was conducted to identify what, if any, effect does the variation of cell type, cell seeding density, initial collagen concentration, and serum concentration have on the rate of initial collagen gel contraction. The collagen-cell suspension was prepared in the identical manner as described above in Example 1. However, the variables in these experiments included cell types of rat aortic smooth muscle cells (SMC) and bovine chordae fibroblasts (BFC), cell suspensions containing 0.5-5 million cells/ml, starting collagen concentrations of 1.0, 2.0 or 3.0 mg/ml, and serum concentrations between 0% and 30%. The collagen/cell suspension, in each experiment, was brought to physiologic pH by addition of 0.1 N NaOH, pipetted into the rectangular shaped well as described above and shown in FIG. 1, and incubated at 37° C. Within 2 hours, a collagen gel formed and attached to the glass wrapped rods positioned at the ends of the well. These rods allowed the shrinkage to occur only transverse to the axis of the well (i.e., Axis A′). After 2-8 weeks of culture, these constructs were examined histologically, biochemically and mechanically.

The rate of initial collagen gel contraction did indeed depend on cell type, cell seeding density, initial collagen concentration and serum concentration. At the end of each experiment, the original transparent gel became a dense, cylindrical construct as shown in FIG. 6I. Gels seeded with rat aorta smooth muscle cells compacted more quickly than those seeded with bovine chordae fibroblasts. The higher the cell seeding density, the faster the collagen gel contracted for both SMC and BCF-seeded gels. Initial collagen concentration influenced gel contraction, particularly at the beginning phase. The greater the initial collagen concentration, the slower was the rate of gel contraction. After culturing for 50 days, all constructs contracted to similar diameters, regardless of cell type. Constructs with zero or low serum concentration (about 2%) contracted to only about 80% of their original diameter, even after 50 days of culture. At that point, they were still gelatinous and unable to hold any loads. Histologic observations demonstrated that culture time greatly affected collagen fiber orientation. After 50 days of culture, the constructs showed a fibrillar orientation similar to that of natural mitral valve chordae.

### EXAMPLE 4

The collagen-cell suspension was prepared in the identical manner as described above in Example 1. However, trace elements (e.g., Mg²⁺, Zn²⁺, Fe²⁺ and Mn²⁺), amino acids (e.g., Met, Cys and α-KG), vitamins (e.g., C and B complex), and growth factors were added to the cell culture medium prepared from DMEM/F12 (1:1) with 10% fetal bovine serum. In control experiments, no supplements were added. After 4 weeks of culture, the constructs were examined histologically, biochemically and mechanically.

The new medium enhanced cell proliferation and collagen synthesis. The optimum concentration of sodium ascorbate was preferably about 100 mg/L and pantothenic acid was about 30 mg/L. This produced constructs twice as strong as controls. The conclusion made was that nutrition fortified and balanced medium provides an effective way to increase the mechanical strength of the constructs.

There have been described and illustrated herein several embodiments of tissue equivalents useful in the repair and replacement of mitral valve chordae, and the method of fabrication thereof While this invention has been described with an emphasis upon preferred embodiments, it will be obvious to those of ordinary skill in the art that variations of the preferred compounds and methods may be used and that it is intended that the invention may be practiced otherwise than as specifically described herein. For example, those skilled in the art will appreciate that certain features of one embodiment may be combined with features of another embodiment to provide yet additional embodiments. It will therefore be appreciated by those skilled in the art that yet other modifications could be made to the invention set forth herein without deviating from its spirit and scope as claimed and described herein.

What is claimed is:

1. A connective tissue equivalent comprised of:
a body comprising collagen fibrils, said body having a proximal portion and a distal portion, said body terminates into at least two ends at said distal portion and said body terminates into at least one end at said proximal end, said body and ends having collagen fibrils oriented along an axis of alignment.

2. The connective tissue equivalent of claim 1, wherein said body further comprises tissue cells embedded within said collagen fibrils.

3. The connective tissue equivalent of claim 1, wherein the distance along said body between one of said at least two ends at said distal portion and said end at said proximal end defines a first path such that a portion of said collagen fibrils are generally oriented along the direction of said first path.

4. The connective tissue equivalent of claim 1, wherein the distance along said body between the other of said at least two ends at said distal portion and said end at said proximal end defines a second path such that a portion of said collagen fibrils are generally oriented along the direction of said second path.

5. The connective tissue equivalent of claim 1, wherein said at least two ends at said distal portion being suitable for attachment to a tissue body.

6. The connective tissue equivalent of claim 5, wherein said tissue body is a leaflet suturing strip.

7. The connective tissue equivalent of claim 1, wherein said end at said proximal portion being suitable for attachment to a tissue body.

8. The connective tissue equivalent of claim 7, wherein said tissue body is a papillary muscle pad.

9. A tissue equivalent comprising:
a construct comprised of collagen fibrils and living tissue cells embedded within said collagen fibrils, said construct including a body that extends into a first arm defining a first path and a second arm defining a second path.

10. The tissue equivalent of claim 9, wherein a portion of said collagen fibrils are generally oriented in a direction substantially parallel to the direction of said first path at any given location along said first path.

11. The tissue equivalent of claim 9, wherein a portion of said collagen fibrils are generally oriented in a direction substantially parallel to the direction of said second path at any given location along said second path.

12. The tissue equivalent of claim 9, wherein said construct includes a proximal portion and a distal portion, said first arm terminates into a first end at said distal portion and said second arm terminates into a second end at said distal portion, said body terminates into a third end at said proximal end.

13. The tissue equivalent of claim 12, wherein said first and second ends at said distal portion being suitable for attachment to a tissue body.

14. The tissue equivalent of claim 13, wherein said tissue body is a leaflet suturing strip.

15. The tissue equivalent of claim 12, wherein said third end at said proximal portion being suitable for attachment to a tissue body.

16. The tissue equivalent of claim 15, wherein said tissue body is a papillary muscle pad.

17. The tissue equivalent of claim 9, furthering comprising a third arm that extends from said body thereby defining a third path.

18. A connective tissue equivalent comprising:
a body having a plurality of arms extending from said body; and
said body and plurality of arms comprised of collagen fibrils having living cells embedded therein.

19. The connective tissue equivalent of claim 18, further comprising a tissue equivalent body in communication with said plurality of arms.

20. The connective tissue equivalent of claim 19, wherein said tissue equivalent body is a leaflet suturing strip.

21. The connective tissue equivalent of claim 18, wherein said body and plurality of arms form a generally Y-shaped portion of said connective tissue equivalent.

22. The connective tissue equivalent of claim 18, further comprising a tissue equivalent body in communication with said body.

23. The connective tissue equivalent of claim 19, wherein said tissue equivalent body is a papillary muscle pad.

24. A tissue engineered mitral valve chordae comprised of:
a construct formed of collagen fibrils, said construct includes a body having a plurality of arms extending therefrom, said construct having mechanical integrity substantially similar to natural chordae.

25. The tissue engineered mitral valve of claim 24, wherein said mechanical integrity is selected from the group consisting of extensibility, stiffness, strength, flexibility, pliability, and combinations thereof.

26. A method of producing a tissue equivalent, comprising:
combining collagen fibrils with living tissue cells to form a collagen/cell mixture;
neutralizing said collagen/cell mixture to form a collagen/cell suspension;
forming a collagen/cell gel by delivering said collagen/cell suspension into a well having a first attaching means and at least two attaching means opposing said attaching means; and
maintaining said collagen/cell gel under conditions that allow contraction of said collagen/cell gel to form a tissue equivalent.

27. The method of claim 26, wherein said first attaching means and one of said at least two attaching means cause said collagen gel to contract in a direction transverse to the direction of attachment defining a first path such that a first portion of collagen fibrils are generally aligned along the direction of the first path.

28. The method of claim 27, wherein said first attaching means and the other of said at least two attaching means cause said collagen gel to contract in a direction transverse to the direction of attachment defining a second path such that a second portion of collagen fibrils are generally aligned along the direction of the second path.

29. The method of claim 26, wherein said attaching means comprises porous anchors suitable for tethering said collagen gel.

30. The method of claim 26, wherein the ratio between the width of the said first attaching means and said at least two attaching means is approximately 2:1.
